(19) United States
(12) Patent Application Publication
(10) Pub. No.: US 2013/0295031 A1
(43) Pub. Date: Nov. 7, 2013

(54) COSMETIC COMPOSITION AND PROCESS FOR CURLING KERATIN FIBRES

(71) Applicants: Guillaume Kergosien, Chaville (FR); Alexandra Schvent, Saint Mande (FR); Nathalie Martin, Viarnes (FR)

(72) Inventors: Guillaume Kergosien, Chaville (FR); Alexandra Schvent, Saint Mande (FR); Nathalie Martin, Viarnes (FR)

(73) Assignee: L'Oreal, Paris (FR)

(21) Appl. No.: 13/883,430

(22) PCT Filed: Oct. 27, 2011
(86) PCT No.: PCT/EP11/68899

§ 371 Date: Jul. 22, 2013

Related U.S. Application Data
(60) Provisional application No. 61/431,101, filed on Jan. 10, 2011.

(30) Foreign Application Priority Data
Nov. 5, 2010 (FR) 1059157

(51) Int. Cl. A61K 8/81; A61Q 5/06; A61Q 1/10; A61Q 5/00

(52) U.S. Cl. CPC A61K 8/8152; A61Q 5/00; A61Q 5/06; A61Q 1/10
USPC 424/70.7; 424/70.11

(57) ABSTRACT

The present invention relates to a cosmetic composition for coating keratin fibres, and especially the eyelashes, comprising: a polymer, derived from the (co-) (homo-)polymerization of (poly)cyclic monomers with a glass transition temperature (Tg) of greater than or equal to 80° C. and a weight-average molecular mass (Mw) of greater than 80 000 g/mol, the said (poly)cyclic monomers comprising (meth)acrylate monomers of formula H₂C═C(R)—C(O)—O—X, with R=H or CH₃, and X corresponding to at least one substituted or unsubstituted cycloalkyl group, a volatile organic solvent present in a content strictly greater than 20%. The present invention also relates to a process for curling keratin fibres.

The present invention relates to a cosmetic composition for coating keratin fibres, and in particular the eyelashes or the eyebrows. In particular, the said cosmetic composition is a composition for making up and/or caring for keratin fibres. The present invention also relates to a process for coating keratin fibres in order to curl them.

The composition used may especially be in the form of an eyelash product such as a mascara, or an eyebrow product. More preferentially, the invention relates to a mascara. The term “mascara” means a composition intended to be applied to the eyelashes: it may be an eyelash makeup composition, an eyelash makeup base (also known as a base coat), a composition to be applied over a mascara, also known as a top coat, or a cosmetic composition for treating the eyelashes. The mascara is more particularly intended for human eyelashes, but also false eyelashes.

Compositions for coating keratin fibres may be formed from at least one wax or a mixture of waxes dispersed in an aqueous liquid or organic solvent phase. They generally have a pasty texture and are conditioned in a container that is closed off by an applicator for the said composition. This applicator may especially be in the form of a brush or a comb configured to take up the product contained in the container and to be placed in contact with the keratin fibres to be made up, such as the eyelashes or the eyebrows.

Compositions for coating keratin fibres may comprise an aqueous liquid phase and a fatty phase dispersed in the said aqueous phase by means of an emulsifying system. The fatty phase may comprise a waxy phase. The emulsifying system may comprise a surfactant.

Patent application WO 2010/014 328 describes compositions comprising particular polymers formulated as emulsions and having contractile properties, enabling the composition, once applied to the eyelash fringe, to curl the eyelashes. However, a drawback associated with such compositions is their sensitivity to water. In particular, the makeup film obtained after applying these compositions may disintegrate in the course of the day, allowing the grains to appear that become deposited around the eyes and form unsightly marks.

Patent application US 2009/0 053 270 discloses a particular polymer (A) used in aqueous phase combining a monomer (a) of methacrylic acid or a methacrylic acid salt with a monomer (b) chosen from several compounds and especially from isobornyl (meth)acrylate.

Patent application US 2009/0 047 308 discloses copolymers comprising at least two monomers containing particular acrylate or methacrylate groups, one having a glass transition temperature (Tg) of less than or equal to 20° C., the other having a Tg of greater than or equal to 20° C.

Patent application US 2010/0 080 763 also discloses particular flexible copolymers with a low Tg, formulated via an aqueous route.

A drawback here also associated with such prior-art compositions is both their sensitivity to water and their excessively low rigidity.

One aim of the present invention lies in the development of a composition for coating keratin fibres and of a process for coating keratin fibres, which improves the curling of the eyelashes.

Another aim of the present invention is to produce a composition for coating keratin fibres that is resistant to water and especially to ambient humidity, sweat, tears and/or sebum.

Unexpectedly, the inventor has discovered that the use of particular polymers formulated in a volatile organic oil makes it possible to obtain a composition which, when applied to the eyelashes, gives them very good curling. The explanation that the inventor gives to this phenomenon is that the formulation of a polymer termed as being rigid, and having both a high glass transition temperature (Tg) and a high molecular mass, in a large amount of volatile organic solvent makes it possible, firstly, to sufficiently plasticize this polymer to allow its deposition onto the eyelashes, and then, secondly, the evaporation of the volatile organic solvent from the composition causes gradual hardening of the film, which once again becomes rigid before total evaporation of the solvent. This property cannot be achieved if the glass transition temperature (Tg) and molecular mass criteria are not satisfied. This capacity to become rigid despite the residual presence of solvent will make it possible, during the evaporation of the remaining solvent, to bring about spontaneous folding of the eyelash. What is more, the fact that this composition is of essentially hydrophobic nature imparts good water resistance thereto and also to all the aqueous or water-soluble compounds that may have a negative impact on the wear property of the deposit (sweat, tears, atmospheric moisture, etc.).

To do this, one subject of the present invention is a cosmetic composition for coating keratin fibres and especially the eyelashes, comprising:
a polymer, derived from the statistical (co-) (homo-)polymerization of (poly)cyclic monomers with a glass transition temperature (Tg) of greater than or equal to 80° C. and a weight-average molecular mass (Mw) of greater than 80 000 g/mol, the said (poly)cyclic monomers comprising (meth)acrylate monomers of formula H₂C═C(R)—C(O)—O—X, with R=H or CH₃, and X corresponding to at least one substituted or unsubstituted cycloalkyl group,
a volatile organic solvent present in a content strictly greater than 20% by weight relative to the total weight of the composition.

For the purposes of the present invention, and unless otherwise indicated, the following definitions apply:
“polymer”, a succession of at least three monomers;
“statistical (co-) (homo-)polymerization”, statistical co-polymerization or homo-polymerization;
“co-polymerization”, the formation of a polymer derived from at least two types of monomer;
“statistical co-polymerization”, the formation of a polymer from two types of monomer arranged randomly such that a statistical polymer in particular obtained by standard radical polymerization of two monomers is characterized by the distribution of the monomers that is not identical on all the chains, and by the length of the said chains that is not identical for all the chains;
“homo-polymerization”, the formation of a polymer derived from only one type of monomer;
“(poly)cyclic monomers”, monocyclic or polycyclic monomers, in particular bicyclic monomers;
“(meth)acrylate monomers”, acrylate or methacrylate monomers;
“cycloalkyl group”, a saturated or unsaturated, fused or non-fused hydrocarbon-based monocyclic or polycyclic group, in particular bicyclic, which is preferably non-aromatic, comprising from 5 to 20 substituted or unsubstituted carbon atoms, where appropriate including the carbon atom(s) constituting the bridge;
“substituted with one or more alkyl groups”, the cycloalkyl group contains one or more carbon atoms that bear a hydrocarbon-based group chosen from optionally substituted C₁-C₁₀ alkyl and optionally substituted C₂-C₁₀ alkenyl;
the “alkyl” radicals are saturated, linear or branched, generally C₁-C₄ hydrocarbon-based radicals such as methyl, ethyl, propyl and butyl;
the “alkenyl” radicals are unsaturated, linear or branched hydrocarbon-based radicals, comprising one or two conjugated or unconjugated double bonds t, the alkenyl group, which is generally C₂-C₅, preferentially comprises only one double bond such as ethylenyl —CH2=CH2-;
“optionally substituted”, the C₁-C₁₀ alkyl or C₂-C₁₀ alkenyl may be substituted with a (hetero)cycloalkyl group, which is preferably non-aromatic;
“rigid”, a cycloalkyl group comprising two non-contiguous carbon atoms forming a bridge between them, the said bridge being:
preferably an alkylene chain of formula —(CRR′)n- with n between 1 and 3 inclusive, preferably equal to 1, and R and R′ being, both or independently of each other, an H atom or a C₁-C₆ alkyl, and preferably both being an H atom or a methyl, or
an alkylene chain of formula —RC═CR′—, R and R′ being, independently of each other, an H atom or a C₁-C₆ alkyl, preferably R and R′ both being an H atom,
“comprises a glass transition temperature of greater than or equal to 80° C.”, the fact that the homopolymer or at least 90% of the constituent monomers of the statistical copolymer has a glass transition temperature of greater than or equal to 80° C.

The said monomers are advantageously sterically hindered.

This cycloalkyl group X is advantageously bridged. It is preferably chosen from isobornyl, dicyclopentyl, dicyclopentenyl, adamantyl and in particular 3,5 dimethyladamantyl, and cyclohexyl and in particular 3,3,5-trimethylcyclohexyl. According to one particular embodiment, the glass transition temperature (Tg) is greater than or equal to 100° C. The said monomers preferably comprise at least one ethylenic unsaturation. Advantageously, these monomers are not aromatic.

Preferably, the weight-average molecular mass (Mw) of the polymer is between 90 000 g/mol and 500 000 g/mol.

Preferably, the weight-average molecular mass (Mw) of the polymer is between 100 000 g/mol and 300 000 g/mol.

According to one particular embodiment, the said polymer may be free of hydrophilic monomer.

This polymer is preferably soluble in the said volatile organic solvent. This polymer advantageously has film-forming properties.

According to one particular embodiment, this polymer is amorphous.

According to one preferred embodiment, the polymer is a copolymer of isobornyl acrylate and of isobornyl methacrylate.

Preferably, the polymer is present in a content of greater than or equal to 20% by weight relative to the total weight of the composition.

Advantageously, the said volatile organic solvent is hydrocarbon-based, containing from 7 to 16 carbon atoms.

According to one preferred embodiment, the said composition has a water content of less than or equal to 10% by weight relative to the total weight of the composition, better still 7%, in particular 5% and preferably 2% by weight, or even is anhydrous.

According to one preferred embodiment, the said composition comprises at least one colouring agent. This colouring agent may be a pigment. This composition may be a makeup composition, a makeup base, a “top coat” composition to be applied over a makeup, or a cosmetic composition for treating or caring for keratin fibres. Preferably, the composition in accordance with the invention is a mascara.

According to a second aspect, a subject of the present invention is also an assembly or kit for coating keratin fibres, comprising:
at least one cosmetic composition for coating keratin fibres as described previously, and
at least one applicator for the composition, the said applicator comprising means, where appropriate in relief, configured to come into contact with the said keratin fibres, such as the eyelashes or the eyebrows, so as to smooth and/or separate the eyelashes or the eyebrows. Such reliefs may comprise teeth, bristles or the like. The said assembly, and in particular the said applicator, may optionally be equipped with means for vibrating and/or heating the said composition.

According to a third aspect, a subject of the present invention is also an assembly or kit for conditioning and applying a composition for coating keratin fibres, comprising:
a device for conditioning the said cosmetic composition for coating keratin fibres as described previously,
an applicator for the said composition.

The said applicator may be integrally attached to a handling member forming a cap for the said conditioning device. In other words, the said applicator may be mounted in a removable position on the said device between a closed position and an open position of a dispensing aperture of the device for conditioning the said composition.

According to a fourth aspect, a subject of the present invention is also a process for coating keratin fibres, and in particular for making up the eyelashes, comprising a step of applying a cosmetic composition for coating keratin fibres as described previously.

According to a fifth aspect, a subject of the present invention is also a process for curling keratin fibres, comprising a step of applying a cosmetic composition for coating keratin fibres as described previously.

Other characteristics, properties and advantages of the present invention will emerge more clearly on reading the description and the examples that follow.

Polymer

The compositions in accordance with the invention comprise at least one polymer derived from the statistical (co-) (homo-)polymerization of (poly)cyclic monomers with a glass transition temperature (Tg) of greater than or equal to 80° C. and a weight-average molecular mass (Mw) of greater than 80 000 g/mol, comprising (meth)acrylate monomers of formula H₂C═C(R)—C(O)—O—X, with R=H or CH₃, and X corresponding to one or more substituted or unsubstituted cycloalkyl groups.

As regards the glass transition temperature of the (poly)cyclic monomers according to the invention, when the polymer is a homopolymer, the monomer unit constituting it has a Tg of greater than or equal to 80° C., and when the polymer according to the invention is a copolymer, at least 90%, preferably at least 95% and preferably all of the at least two types of monomer constituting it each have a Tg of greater than or equal to 80° C.

The term “monomer with a Tg of greater than or equal to 80° C.” means a monomer whose homopolymer would lead to a Tg of greater than or equal to 80° C.

The polymer obtained is advantageously predominantly derived from this statistical (co-) (homo-)polymerization of (poly)cyclic monomers. This means that the polymer according to the invention is obtained to more than 70%, better still 90% or more, by statistical homopolymerization or copolymerization of the said monocyclic or polycyclic monomers.

The polymers may have a linear structure or a non-linear structure, for instance a copolymer of branched, radial or star structure.

Preferably, the polymers according to the invention are chosen from amorphous polymers. The term “amorphous polymer” means a polymer that does not have a crystalline form.

Preferably, the polymers according to the invention are in solution in the said volatile organic solvent. These polymers according to the invention preferably have film-forming properties. In the present patent application, the term “film-forming polymer” means a polymer that is capable, by itself or in the presence of an auxiliary film-forming agent, of forming a macroscopically continuous film that adheres to the eyelashes, and preferably a cohesive film, better still a film whose cohesion and mechanical properties are such that the said film can be isolated and manipulated individually, for example when the said film is prepared by pouring onto a non-stick surface such as a Teflon-coated or silicone-coated surface.

Preferably, the polymers according to the invention have a glass transition temperature (Tg) of greater than or equal to 100° C. Advantageously, the said polymers have a glass transition temperature (Tg) of between 80 and 160° C.

The polymers preferably have a low mass polydispersity and preferably a low composition polydispersity. The mass polydispersity may be illustrated by means of the mass polydispersity index (Ip) of the polymer, which is equal to the ratio of the weight-average molecular mass (Mw) to the number-average molecular mass (Mn). A low mass dispersity reflects approximately identical chain lengths, which is the case for the polymers according to the present invention. Preferably, the polymer according to the invention has a mass polydispersity index of less than or equal to 3, preferably between 1.1 and 2.5, especially between 1.15 and 2.3, or even between 1.2 and 2.0, or 1.9 or even 1.8.

The polymers have a weight-average molecular mass (Mw) of greater than 80 000 g/mol. Advantageously, this weight-average molecular mass is between 90 000 and 500 000 g/mol and better still between 100 000 and 300 000 g/mol.

Preferably, the polymers have a number-average molecular mass (Mn) of greater than 50 000 g/mol. Advantageously, this number-average molecular mass is between 50 000 and 500 000 g/mol and better still between 50 000 and 300 000 g/mol.

The weight-average molecular mass (Mw) and number-average molecular mass (Mn) are determined by gel permeation liquid chromatography (GPC), eluting with THF, calibration curve established with linear polystyrene standards, refractometric detector.

The polymers according to the invention also preferentially have a low composition dispersity. This means that all the polymer chains have an approximately analogous composition (i.e. sequence of monomers) and are therefore of homogeneous composition. In order to show that all the polymer chains have an analogous composition, use will advantageously be made of liquid adsorption chromatography (or LAC), which makes it possible to separate the polymer chains, not according to their molecular weight, but according to their polarity. This criterion reflects the chemical composition of the polymers constituting the material, the monomers being known. Reference may be made to the publication Macromolecules (2001), 34, 2667, which describes the LAC technique.

The composition polydispersity may especially be defined from the adsorption chromatography (LAC) curve (curve representing the proportion of polymers as a function of the elution volume): if we take “V^{1/2} min” as the minimum value of the elution volume at mid-height of the curve, and “V^{1/2} max” as the maximum value of the elution volume at mid-height of the curve, the composition polydispersity is generally considered as low if the difference (V^{1/2} max−V^{1/2} min) is less than or equal to 3.5, especially between 1 and 2.8 and better still between 1.2 and 2.5.

Moreover, the LAC curve may be defined by a Gaussian curve of formula:

$$y = \frac{A}{w \sqrt{\frac{π}{2}}} \times {e}^{- 2 \frac{{( x - {x}_{0} )}^{2}}{{w}^{2}}} + {y}_{0}$$

in which:
x₀ is the value of x (elution volume) at the centre of the peak
w is equal to twice the standard deviation of the Gaussian distribution (i.e. 2σ) or alternatively corresponds to approximately 0.849 times the width of the peak at mid-height
A represents the area under the peak
y₀ is the value of y corresponding to x₀.

The composition dispersity of the polymers according to the invention may also be defined by the value of w defined above. Preferably, the said value w is between 1 and 3, especially between 1.1 and 2.3 and better still between 1.1 and 2.0.

Advantageously, the polymers do not comprise any hydrophilic or polar monomers. For the purposes of the present invention, the term “polar monomers” means monomers whose solubility parameter at 25° C., δₐ, is other than 0 (J/cm³)^{1/2}. In particular, the term “polar monomer” means a compound whose chemical structure is formed essentially from, or even constituted by, carbon and hydrogen atoms, and comprising at least one highly electronegative heteroatom such as an oxygen, nitrogen or phosphorus atom. The definition and calculation of the solubility parameters in the Hansen three-dimensional solubility space are described in the article by C. M. Hansen: “The three dimensional solubility parameters”, J. Paint Technol. 39, 105 (1967).

According to this Hansen space:
δ_{D} characterizes the London dispersion forces derived from the formation of dipoles induced during molecular impacts;
δₚ characterizes the Debye interaction forces between permanent dipoles and also the Keesom interaction forces between induced dipoles and permanent dipoles;
δₕ characterizes the specific interaction forces (such as hydrogen bonding, acid/base, donor/acceptor, etc.); and
δₐ is determined by the equation: δₐ=(δₚ²+δₕ²)½.

The parameters δₚ, δₕ, δ_{D} and δₐ are expressed in (J/cm³)^{1/2}.

Preferably, the polymers obtained comprise less than 10%, preferably less than 5%, and preferably are free, of acrylic acid, methacrylic acid, and/or salts thereof, relative to the total weight of the polymers.

The said cycloalkyl group is preferably chosen from isobornyl, cyclohexyl, dicyclopentyl, dicyclopentenyl and adamantyl.

The cycloalkyl group according to the invention may be substituted or unsubstituted. In particular, it may be substituted with one or more hydrocarbon-based groups, for example alkyl or alkenyl groups.

The alkyl group(s) preferably comprise from 1 to 10 carbon atoms. Preferably, this alkyl group is a methyl or an ethyl group.

The alkenyl group(s) preferably comprise from 2 to 10 carbon atoms. Preferably, this alkenyl group is an ethylene.

The substituted cycloalkyl group is preferably chosen from 3,5-dimethyladamantyl and 3,3,5-trimethylcyclohexyl.

The cycloalkyl group is preferably bridged. Preferably, it comprises one or more unsaturations. Preferably, the group is non-aromatic.

According to one preferred embodiment, the polymers of the invention comprise at least one monomer chosen from isobornyl acrylate and isobornyl methacrylate, or a mixture of these two monomers.

The (meth)acrylate polymers according to the invention may be derived, for example, from the statistical homopolymerization or copolymerization of monomers:
of formula H₂C═C(R)—C(O)—O—X, with R=H or CH₃, and X corresponding to a substituted or unsubstituted bridged cycloalkyl group, comprising from 7 to 20 carbon atoms and preferably comprising from 7 to 12 carbon atoms. The following homopolymers are most particularly preferred:

isobornyl acrylate

isobornyl methacrylate

dicyclopentenyl acrylate

dicyclopentenyl methacrylate

dicyclopentyl acrylate

dicyclopentyl methacrylate

adamantyl acrylate

adamantyl methacrylate

3,5-dimethyladamantyl acrylate

3,5-dimethyladamantyl methacrylate

and also the copolymers derived from the mixture of at least two of these monomers, for example isobornyl acrylate and isobornyl methacrylate.
of formula CH₂—C(CH₃)—C(O)—O—X, with R=CH₃ and X corresponding to a substituted or unsubstituted cyclohexyl group.

The following homopolymers are most particularly preferred:

cyclohexyl methacrylate

3,3,5-trimethylcyclohexyl methacrylate

and also the copolymers derived from the mixture of these monomers.

Preference is also given to copolymers comprising:

isobornyl acrylate and isobornyl methacrylate;

isobornyl acrylate, isobornyl methacrylate and butyl acrylate;

isobornyl acrylate, isobornyl methacrylate and butyl methacrylate;

isobornyl acrylate, isobornyl methacrylate and isobutyl acrylate;

isobornyl acrylate, isobornyl methacrylate and isobutyl methacrylate;

isobornyl methacrylate and butyl acrylate;

isobornyl methacrylate and butyl methacrylate;

isobornyl methacrylate and isobutyl acrylate;

isobornyl methacrylate and isobutyl methacrylate;

dicyclopentenyl acrylate and dicyclopentenyl methacrylate;

dicyclopentyl acrylate and dicyclopentyl methacrylate;

and mixtures thereof.

The polymers according to the invention are preferably present in a content of greater than or equal to 20%, better still greater than or equal to 25%, or even 30%, by weight of solids relative to the total weight of the composition. They may in particular be present in a content ranging from 26% to 40% by weight and preferably ranging from 28% to 36% by weight relative to the total weight of the composition.

Oil or Organic Solvent

The compositions according to the invention comprise at least one oil or organic solvent. The term “oil” means a fatty substance that is liquid at room temperature and at atmospheric pressure. The compositions according to the invention more specifically comprise at least one volatile organic oil. According to one particular embodiment, the composition according to the invention may comprise, besides the said at least one volatile oil, at least one non-volatile oil.

Volatile Oil

The composition according to the invention comprises at least one volatile oil. The volatile oil is present in a content strictly greater than 20% by weight relative to the total weight of the composition. It may in particular be present in a content ranging from 25% to 90% by weight and preferably from 30% to 70% by weight relative to the total weight of the composition.

The term “volatile oil” means an oil (or non-aqueous medium) that is capable of evaporating on contact with the skin or the keratin fibre in less than one hour, at room temperature and atmospheric pressure. The volatile oil is a volatile cosmetic oil, which is liquid at room temperature, with an evaporation rate of between 0.01 and 200 mg/cm²/min, limits inclusive.

To measure this evaporation rate, 15 g of oil or of oil mixture to be tested are placed in a crystallizing dish 7 cm in diameter, placed on a balance that is in a large temperature-regulated chamber of about 0.3 m³, at a temperature of 25° C., and hygrometry-regulated, at a relative humidity of 50%. The liquid is allowed to evaporate freely, without stirring it, while providing ventilation by means of a fan (Papst-Motoren, reference 8550 N, rotating at 2700 rpm) placed in a vertical position above the crystallizing dish containing the said oil or the said mixture, the blades being directed towards the crystallizing dish, 20 cm away from the bottom of the crystallizing dish. The mass of oil remaining in the crystallizing dish is measured at regular intervals. The evaporation rates are expressed in mg of oil evaporated per unit area (cm²) and per unit of time (minutes).

This volatile oil is preferably a hydrocarbon-based oil.

The hydrocarbon-based volatile oil is preferably chosen from hydrocarbon-based oils containing from 7 to 16 carbon atoms.

The composition according to the invention may contain one or more volatile branched alkane(s). The term “one or more volatile branched alkane(s)” means, without preference, “one or more volatile branched alkane oil(s)”.

As volatile hydrocarbon-based oils containing from 7 to 16 carbon atoms, mention may be made especially of C8-C16 branched alkanes, for instance C8-C16 isoalkanes (also known as isoparaffins), isododecane, isodecane, isohexadecane and for example the oils sold under the trade names Isopar or Permethyl, C8-C16 branched esters such as isohexyl neopentanoate, and mixtures thereof. Preferably, the volatile hydrocarbon-based oil containing from 8 to 16 carbon atoms is chosen from isododecane, isodecane and isohexadecane, and mixtures thereof, and is especially isododecane.

The composition according to the invention may contain one or more volatile linear alkane(s). The term “one or more volatile linear alkane(s)” means, without preference, “one or more volatile linear alkane oil(s)”.

A volatile linear alkane that is suitable for use in the invention is liquid at room temperature (about 25° C.) and at atmospheric pressure (760 mmHg).

The term “volatile linear alkane” that is suitable for use in the invention means a cosmetic linear alkane, which is capable of evaporating on contact with the skin in less than one hour, at room temperature (25° C.) and atmospheric pressure (760 mmHg, i.e. 101 325 Pa), which is liquid at room temperature, especially having an evaporation rate ranging from 0.01 to 15 mg/cm²/minute, at room temperature (25° C.) and atmospheric pressure (760 mmHg).

The linear alkanes, preferably of plant origin, comprise from 7 to 15 carbon atoms, in particular from 9 to 14 carbon atoms and more particularly from 11 to 13 carbon atoms.

As examples of linear alkanes that are suitable for use in the invention, mention may be made of the alkanes described in patents WO 2007/068 371 or WO 2008/155 059 of the company Cognis (mixtures of different alkanes differing by at least one carbon). These alkanes are obtained from fatty alcohols, which are themselves obtained from coconut oil or palm oil.

As examples of linear alkanes that are suitable for use in the invention, mention may be made of n-heptane (C7), n-octane (C8), n-nonane (C9), n-decane (C10), n-undecane (C11), n-dodecane (C12), n-tridecane (C13), n-tetradecane (C14) and n-pentadecane (C15), and mixtures thereof, and in particular the mixture of n-undecane (C11) and n-tridecane (C13) described in Example 1 of patent application WO 2008/155 059 from the company Cognis. Mention may also be made of n-dodecane (C12) and n-tetradecane (C14) sold by Sasol under the references, respectively, Parafol 12-97 and Parafol 14-97, and also mixtures thereof.

The linear alkane may be used alone or as a mixture of at least two distinct alkanes differing from each other by a carbon number of at least 1, and especially a mixture of at least two distinct linear alkanes comprising from 10 to 14 carbon atoms, differing from each other by a carbon number of at least 2, and in particular a mixture of C11/C13 volatile linear alkanes or a mixture of C12/C14 linear alkanes, in particular an n-undecane/n-tridecane mixture (such a mixture may be obtained according to Example 1 or Example 2 of WO 2008/155 059).

As a variant or additionally, the composition prepared may comprise at least one volatile silicone oil or solvent that is compatible with cosmetic use.

The term “silicone oil” means an oil containing at least one silicon atom, and especially containing Si—O groups. According to one embodiment, the said composition comprises less than 10% by weight of non-volatile silicone oil(s), relative to the total weight of the composition, better still less than 5% by weight, or even is free of silicone oil.

Volatile silicone oils that may be mentioned include cyclic polysiloxanes and linear polysiloxanes, and mixtures thereof. Volatile linear polysiloxanes that may be mentioned include hexamethyldisiloxane, octamethyltrisiloxane, decamethyltetrasiloxane, tetradecamethylhexasiloxane and hexadecamethylheptasiloxane. Volatile cyclic polysiloxanes that may be mentioned include hexamethylcyclotrisiloxane, octamethylcyclotetrasiloxane, decamethylcyclopentasiloxane and dodecamethylcyclohexasiloxane.

Non-Volatile Oil

The composition according to the invention may comprise at least one non-volatile oil.

In contrast, the term “non-volatile oil” means an oil that remains on the skin or the keratin fibre at room temperature and atmospheric pressure. More specifically, a non-volatile oil has an evaporation rate strictly less than 0.01 mg/cm²/min.

The said at least one non-volatile oil that is suitable for use in the present invention may be chosen from hydrocarbon-based oils and silicone oils.

Non-volatile hydrocarbon-based oils that are suitable for use in the present invention may be chosen in particular from:
hydrocarbon-based oils of plant origin, such as triglycerides formed from fatty acid esters of glycerol, the fatty acids of which may have varied chain lengths from C4 to C28, these chains possibly being linear or branched, and saturated or unsaturated; these oils are especially wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, sesame seed oil, marrow oil, rapeseed oil, blackcurrant oil, evening primrose oil, millet oil, barley oil, quinoa oil, rye oil, safflower oil, candlenut oil, passion flower oil and musk rose oil; or alternatively caprylic/capric acid triglycerides such as those sold by the company Stéarineries Dubois or those sold under the names Miglyol 810®, 812® and 818® by the company Sasol,
synthetic ethers containing from 10 to 40 carbon atoms;
linear or branched hydrocarbons of mineral or synthetic origin, such as petroleum jelly, polydecenes, hydrogenated polyisobutene such as Parleam, and squalane, and mixtures thereof;
synthetic esters such as oils of formula R1COOR2 in which R1 represents a linear or branched fatty acid residue containing from 1 to 40 carbon atoms and R2 represents an in particular branched hydrocarbon-based chain containing from 1 to 40 carbon atoms, on condition that R1+R≧10, for instance purcellin oil (cetostearyl octanoate), isopropyl myristate, isopropyl palmitate, C12-C15 alkyl benzoate, hexyl laurate, diisopropyl adipate, isononyl isononanoate, 2-ethylhexyl palmitate, isostearyl isostearate, alkyl or polyalkyl octanoates, decanoates or ricinoleates such as propylene glycol dioctanoate; hydroxylated esters such as isostearyl lactate and diisostearyl malate; and pentaerythritol esters;
fatty alcohols that are liquid at room temperature, with a branched and/or unsaturated carbon-based chain containing from 12 to 26 carbon atoms, for instance octyldodecanol, isostearyl alcohol, oleyl alcohol, 2-hexyldecanol, 2-butyloctanol and 2-undecylpentadecanol;

higher fatty acids such as oleic acid, linoleic acid or linolenic acid, and mixtures thereof.

The non-volatile silicone oils that are suitable for use in the present invention may be chosen in particular from:

the non-volatile silicone oils that may be used in the composition in accordance with the invention may be non-volatile polydimethylsiloxanes (PDMSs), polydimethylsiloxanes comprising alkyl or alkoxy groups, that are pendent and/or at the end of a silicone chain, the groups each containing from 2 to 24 carbon atoms, phenyl silicones, for instance phenyl trimethicones, phenyl dimethicones, phenyltrimethylsiloxydiphenylsiloxanes, diphenyl dimethicones, diphenylmethyldiphenyltrisiloxanes and 2-phenylethyl trimethylsiloxysilicates.

The content of non-volatile oil or organic solvent in the composition may range, for example, from 0.1% to 20% by weight and in particular from 1% to 5% by weight relative to the total weight of the said composition.

Waxes

The composition preferably comprises at least one wax.

The term “wax” is understood, within the meaning of the present invention, to mean a lipophilic compound, which is solid at room temperature (25° C.), with a reversible solid/liquid change of state, which has a melting point of greater than or equal to 30° C., which may be up to 120° C.

The melting point of the wax may be measured using a differential scanning calorimeter (DSC), for example the calorimeter sold under the name DSC 30 by the company Mettler.

The waxes may be hydrocarbon-based waxes, fluoro waxes and/or silicone waxes, and may be of plant, mineral, animal and/or synthetic origin. In particular, the waxes have a melting point of greater than 25° C. and better still greater than 45° C.

Hydrocarbon-based waxes, for instance beeswax, lanolin wax or Chinese insect wax; rice wax, carnauba wax, candelilla wax, ouricury wax, esparto grass wax, cork fibre wax, sugar cane wax, Japan wax and sumach wax; montan wax, microcrystalline waxes, paraffins and ozokerite; polyethylene waxes, the waxes obtained by Fischer-Tropsch synthesis and waxy copolymers, and also esters thereof, may especially be used.

Mention may also be made of waxes obtained by catalytic hydrogenation of animal or plant oils containing linear or branched C8-C32 fatty chains.

Among these waxes that may especially be mentioned are hydrogenated jojoba oil, isomerized jojoba oil such as the trans-isomerized partially hydrogenated jojoba oil manufactured or sold by the company Desert Whale under the commercial reference Iso-Jojoba-50®, hydrogenated sunflower oil, hydrogenated castor oil, hydrogenated coconut oil, hydrogenated lanolin oil and bis(1,1,1-trimethylolpropane)tetrastearate sold under the name Hest 2T-4S by the company Heterene, bis(1,1,1-trimethylolpropane)tetrabehenate sold under the name Hest 2T-4B by the company Heterene.

Mention may also be made of silicone waxes, for instance alkyl or alkoxy dimethicones containing from 16 to 45 carbon atoms, and fluoro waxes.

The wax obtained by hydrogenation of olive oil esterified with stearyl alcohol, sold under the name Phytowax Olive 18L57 or else the waxes obtained by hydrogenation of castor oil esterified with cetyl alcohol sold under the names Phytowax ricin 16L64 and 22L73 by the company Sophim may also be used. Such waxes are described in patent application FR-A-2 792 190.

The composition may comprise at least one polar wax. The term “polar wax” means waxes comprising in their chemical structure, in addition to carbon and hydrogen atoms, at least one highly electronegative heteroatom, such as O, N or P.

Preferably, the wax is chosen from carnauba wax, candelilla wax, natural (or blanched) beeswax and synthetic beeswax. A synthetic beeswax that may be mentioned is the wax sold under the name Cyclochem 326 A by Evonik Goldschmidt (INCI name: Synthetic Beeswax).

The composition may comprise at least one wax having a hardness ranging from 0.05 MPa to 15 MPa and preferably ranging from 6 MPa to 15 MPa. The hardness is determined by measuring the compression force, measured at 20° C. using the texturometer sold under the name TA-TX2i by the company Rheo, equipped with a stainless-steel cylindrical spindle 2 mm in diameter, travelling at a measuring speed of 0.1 mm/second, and penetrating the wax to a penetration depth of 0.3 mm.

According to one particular embodiment, the compositions according to the invention may comprise at least one wax referred to as a tacky wax, i.e. a wax with a tack of greater than or equal to 0.7 N.s and a hardness of less than or equal to 3.5 MPa.

Using a tacky wax may especially make it possible to obtain a cosmetic composition that applies easily to the eyelashes, attaches well to the eyelashes and leads to the formation of a smooth, uniform and thickening makeup result.

The tacky wax used may especially have a tack ranging from 0.7 N.s to 30 N.s, in particular greater than or equal to 1 N.s, especially ranging from 1 N.s to 20 N.s, in particular greater than or equal to 2 N.s, especially ranging from 2 N.s to 10 N.s and in particular ranging from 2 N.s to 5 N.s.

The tack of the wax is determined by measuring the change in force (compression force or stretching force) as a function of time, at 20° C., using the texturometer sold under the name TA-TX2i® by Rheo, equipped with a conical acrylic polymer spindle forming an angle of 45°.

The measuring protocol is as follows:

The wax is melted at a temperature equal to the melting point of the wax +10° C. The molten wax is poured into a container 25 mm in diameter and 20 mm deep. The wax is recrystallized at room temperature (25° C.) for 24 hours such that the surface of the wax is flat and smooth, and the wax is then stored for at least 1 hour at 20° C. before measuring the tack.

The texturometer spindle is displaced at a speed of 0.5 mm/s, then penetrates the wax to a penetration depth of 2 mm. When the spindle has penetrated the wax to a depth of 2 mm, the spindle is held still for 1 second (corresponding to the relaxation time) and is then withdrawn at a speed of 0.5 mm/s.

During the relaxation time, the force (compression force) decreases greatly until it becomes zero, and then, during the withdrawal of the spindle, the force (stretching force) becomes negative and then rises again to the value 0. The tack corresponds to the integral of the curve of the force as a function of time for the part of the curve corresponding to negative values of the force (stretching force). The tack value is expressed in N.s.

The tacky wax that may be used generally has a hardness of less than or equal to 3.5 MPa, in particular ranging from 0.01 MPa to 3.5 MPa, especially ranging from 0.05 MPa to 3 MPa or even ranging from 0.1 MPa to 2.5 MPa.

The hardness is measured according to the protocol described previously.

Tacky waxes that may be used include a C20-C40 alkyl (hydroxystearyloxy)stearate (the alkyl group comprising from 20 to 40 carbon atoms), alone or as a mixture, in particular a C20-C40 alkyl 12-(12′-hydroxystearyloxy)stearate, of formula (II):

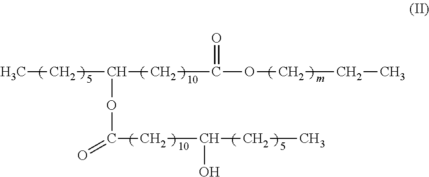

in which m is an integer ranging from 18 to 38, or a mixture of compounds of formula (II).

Such a wax is especially sold under the names Kester Wax K 82 P® and Kester Wax K 80 P® by the company Koster Keunen.

The waxes mentioned above generally have a starting melting point of less than 45° C.

Use may also be made of the microcrystalline wax sold under the reference SP18 by Strahl and Pitsch, which has a hardness of around 0.46 MPa and a tack value of around 1 N.s.

The wax(es) may be present in the form of an aqueous wax microdispersion. The term “aqueous wax microdispersion” means an aqueous dispersion of wax particles, in which the size of said wax particles is less than or equal to about 1 μm.

Wax microdispersions are stable dispersions of colloidal wax particles, and are described especially in Microemulsions Theory and Practice, L. M. Prince Ed., Academic Press (1977) pages 21-32.

In particular, these wax microdispersions may be obtained by melting the wax in the presence of a surfactant, and optionally of some of the water, followed by gradual addition of hot water with stirring. Intermediate formation of an emulsion of the water-in-oil type is observed, followed by a phase inversion with final production of a microemulsion of the oil-in-water type. On cooling, a stable microdispersion of solid colloidal wax particles is obtained.

The wax microdispersions may also be obtained by stirring the mixture of wax, surfactant and water using an agitation means such as ultrasonic waves, a high-pressure homogenizer or turbomixers.

The particles of the wax microdispersion preferably have mean sizes of less than 1 μm (especially ranging from 0.02 μm to 0.99 μm), preferably less than 0.5 μm (especially ranging from 0.06 μm to 0.5 μm).

These particles are formed essentially from a wax or a mixture of waxes. They may, however, comprise a minor proportion of oily and/or pasty fatty additives, a surfactant and/or a common additive/fat-soluble active agent.

The wax may be present in a content at least equal to 10% by weight. Preferably, it is present in a content ranging from 15% to 40% by weight, better still from 16% to 35% by weight and even better still from 16% to 30% by weight relative to the total weight of the composition. The wax may be present in the composition in a content ranging from 0.1% to 50% by weight, preferably ranging from 1% to 40% by weight and preferentially ranging from 5% to 30% by weight, relative to the total weight of the composition.

Additional Lipophilic Film-Forming Polymer

The compositions according to the present patent application may also comprise at least one additional lipophilic film-forming polymer.

In general, the content of “additional lipophilic film-forming polymer” solids in the compositions according to the present patent application ranges from 0.1% to 40%, preferably from 0.5% to 30% and better still from 1% to 20% by weight relative to the total weight of the composition.

The additional lipophilic film-forming polymer may be in solution or in dispersion in the organic solvent.

Among the additional film-forming polymers that may be used in the composition of the present invention, mention may be made of synthetic polymers, of free-radical type or of polycondensate type, and mixtures thereof.

In particular, the weight ratio (by weight of solids) of the polymer according to the invention to the additional lipophilic film-forming polymer(s) may range from 0.1 to 10 and in particular from 1 to 5.

Radical Film-Forming Polymers

The expression “radical film-forming polymer” means a polymer obtained by polymerization of unsaturated and especially ethylenically unsaturated monomers, each monomer being capable of homopolymerizing (unlike polycondensates).

The film-forming polymers of free-radical type may especially be vinyl polymers or copolymers, especially acrylic polymers.

The vinyl film-forming polymers may result from the polymerization of ethylenically unsaturated monomers containing at least one acidic group and/or esters of these acidic monomers and/or amides of these acidic monomers.

Monomers bearing an acidic group which may be used are α,β-ethylenic unsaturated carboxylic acids such as acrylic acid, methacrylic acid, crotonic acid, maleic acid or itaconic acid. (Meth)acrylic acid and crotonic acid are preferably used, and more preferably (meth)acrylic acid.

The esters of acidic monomers are advantageously chosen from (meth)acrylic acid esters (also known as (meth)acrylates), especially (meth)acrylates of an alkyl, in particular of a C₁-C₃₀ and preferably C₁-C₂₀ alkyl, (meth)acrylates of an aryl, in particular of a C₆-C₁₀ aryl, and (meth)acrylates of a hydroxyalkyl, in particular of a C₂-C₆ hydroxyalkyl.

Among the alkyl (meth)acrylates that may be mentioned are methyl methacrylate, ethyl methacrylate, butyl methacrylate, isobutyl methacrylate, 2-ethylhexyl methacrylate, lauryl methacrylate and cyclohexyl methacrylate.

Among the hydroxyalkyl (meth)acrylates that may be mentioned are hydroxyethyl acrylate, 2-hydroxypropyl acrylate, hydroxyethyl methacrylate and 2-hydroxypropyl methacrylate.

Among the aryl (meth)acrylates that may be mentioned are benzyl acrylate and phenyl acrylate.

The (meth)acrylic acid esters that are particularly preferred are the alkyl (meth)acrylates.

According to the present invention, the alkyl group of the esters may be either fluorinated or perfluorinated, i.e. some or all of the hydrogen atoms of the alkyl group are substituted with fluorine atoms.

Examples of amides of the acid monomers that may be mentioned are (meth)acrylamides, and especially N-alkyl(meth)acrylamides, in particular of a C₂-C₁₂ alkyl. Among the N-alkyl(meth)acrylamides that may be mentioned are N-ethylacrylamide, N-t-butylacrylamide, N-t-octylacrylamide and N-undecylacrylamide.

The vinyl film-forming polymers may also result from the homopolymerization or copolymerization of monomers chosen from vinyl esters and styrene monomers. In particular, these monomers may be polymerized with acid monomers and/or esters thereof and/or amides thereof, such as those mentioned previously.

Examples of vinyl esters that may be mentioned are vinyl acetate, vinyl neodecanoate, vinyl pivalate, vinyl benzoate and vinyl t-butylbenzoate.

Styrene monomers that may be mentioned include styrene and α-methylstyrene.

Film-Forming Polycondensates

Among the film-forming polycondensates that may be mentioned are polyurethanes, polyesters, polyester-amides, polyamides, epoxyester resins and polyureas.

The polyurethanes may be chosen from anionic, cationic, nonionic and amphoteric polyurethanes, polyurethane-acrylics, polyurethane-polyvinylpyrrolidones, polyester-polyurethanes, polyether-polyurethanes, polyureas and polyurea-polyurethanes, and mixtures thereof.

The polyesters may be obtained, in a known manner, by polycondensation of dicarboxylic acids with polyols, in particular diols.

The dicarboxylic acid may be aliphatic, alicyclic or aromatic. Examples of such acids that may be mentioned include: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norbornanedicarboxylic acid, diglycolic acid, thiodipropionic acid, 2,5-naphthalenedicarboxylic acid, 2,6-naphthalenedicarboxylic acid. These dicarboxylic acid monomers may be used alone or as a combination of at least two dicarboxylic acid monomers. Among these monomers, the ones preferentially chosen are phthalic acid, isophthalic acid and terephthalic acid.

The diol may be chosen from aliphatic, alicyclic and aromatic diols. The diol used is preferably chosen from: ethylene glycol, diethylene glycol, triethylene glycol, 1,3-propanediol, cyclohexanedimethanol and 4-butanediol. Other polyols that may be used are glycerol, pentaerythritol, sorbitol and trimethylolpropane.

The polyesteramides may be obtained in a manner analogous to that of the polyesters, by polycondensation of diacids with diamines or amino alcohols. Diamines that may be used are ethylenediamine, hexamethylenediamine and meta- or para-phenylenediamine. An amino alcohol that may be used is monoethanolamine.

According to one embodiment variant of the composition according to the invention, the lipophilic film-forming polymer may be a polymer dissolved in a liquid fatty phase comprising oils such as those described previously (the film-forming polymer is then said to be a liposoluble polymer). Preferably, the liquid fatty phase comprises a volatile oil, optionally mixed with a non-volatile oil, the oils possibly being chosen from the oils mentioned above.

Examples of fat-soluble polymers that may be mentioned are copolymers of vinyl ester (the vinyl group being directly linked to the oxygen atom of the ester group and the vinyl ester containing a saturated, linear or branched hydrocarbon-based radical of 1 to 19 carbon atoms, linked to the carbonyl of the ester group) and of at least one other monomer which may be a vinyl ester (other than the vinyl ester already present), an α-olefin (containing from 8 to 28 carbon atoms), an alkyl vinyl ether (in which the alkyl group comprises from 2 to 18 carbon atoms) or an allylic or methallylic ester (containing a saturated, linear or branched hydrocarbon-based radical of 1 to 19 carbon atoms, linked to the carbonyl of the ester group).

These copolymers may be crosslinked with the aid of crosslinking agents, which may be either of the vinyl type or of the allylic or methallylic type, such as tetraallyloxyethane, divinylbenzene, divinyl octanedioate, divinyl dodecanedioate and divinyl octadecanedioate.

Examples of these copolymers that may be mentioned include the following copolymers: vinyl acetate/allyl stearate, vinyl acetate/vinyl laurate, vinyl acetate/vinyl stearate, vinyl acetate/octadecyl vinyl ether, vinyl propionate/allyl laurate, vinyl propionate/vinyl laurate, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethylpropionate/vinyl stearate, allyl dimethylpropionate/vinyl stearate, vinyl propionate/vinyl stearate, crosslinked with 0.2% divinylbenzene, vinyl dimethylpropionate/vinyl laurate, crosslinked with 0.2% divinylbenzene, vinyl acetate/octadecyl vinyl ether, crosslinked with 0.2% tetraallyloxyethane, vinyl acetate/allyl stearate, crosslinked with 0.2% divinylbenzene, vinyl acetate/1-octadecene crosslinked with 0.2% divinylbenzene, and allyl propionate/allyl stearate, crosslinked with 0.2% divinylbenzene.

According to one particular mode, the composition of the invention comprises, besides the copolymers of alkene and of vinyl acetate, at least one liposoluble polymer. In particular, the said liposoluble polymer is chosen from copolymers of a vinyl ester (the vinyl group being directly linked to the oxygen atom of the ester group and the vinyl ester bearing a saturated, linear or branched hydrocarbon-based radical of 1 to 19 carbon atoms, linked to the carbonyl of the ester group) and of an allylic or methallylic ester (bearing a saturated, linear or branched hydrocarbon-based radical of 1 to 19 carbon atoms, linked to the carbonyl of the ester group).

Preferably, the said liposoluble polymer is chosen from vinyl acetate/allyl stearate copolymers.

Examples of liposoluble film-forming polymers that may also be mentioned are liposoluble copolymers, and in particular those resulting from the copolymerization of vinyl esters containing from 9 to 22 carbon atoms or of alkyl acrylates or methacrylates, and alkyl radicals containing from 10 to 20 carbon atoms.

Such liposoluble copolymers may be chosen from copolymers of polyvinyl stearate, polyvinyl stearate crosslinked with the aid of divinylbenzene, of diallyl ether or of diallyl phthalate, copolymers of polystearyl (meth)acrylate, polyvinyl laurate and polylauryl (meth)acrylate, it being possible for these poly(meth)acrylates to be crosslinked with the aid of ethylene glycol dimethacrylate or tetraethylene glycol dimethacrylate.

The liposoluble copolymers defined above are known and are described in particular in patent application FR-A-2 232 303; they may have a weight-average molecular weight ranging from 2000 to 500 000 and preferably from 4000 to 200 000.

According to another mode, at least one copolymer resulting from the copolymerization of vinyl esters containing from 9 to 22 carbon atoms, and in particular copolymers of polyvinyl laurate, may be used as additional liposoluble polymer.

According to one particular mode, the composition according to the invention will comprise, besides the copolymers of alkene and of vinyl acetate, at least one liposoluble polymer chosen from vinyl acetate/allyl stearate copolymers and polyvinyl laurate copolymers, and mixtures thereof.

As liposoluble film-forming polymers that may be used in the invention, mention may also be made of polyalkylenes and in particular copolymers of C2-C20 alkenes, such as polybutene, alkylcelluloses with a linear or branched, saturated or unsaturated C1-C8 alkyl radical, for instance ethylcellulose and propylcellulose, copolymers of vinylpyrrolidone (VP) and in particular copolymers of vinylpyrrolidone and of C2 to C40 and better still C3 to C20 alkene. As examples of VP copolymers which may be used in the invention, mention may be made of the copolymers of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene or VP/acrylic acid/lauryl methacrylate.

Mention may also be made of silicone resins, which are generally soluble or swellable in silicone oils, which are crosslinked polyorganosiloxane polymers. The nomenclature of silicone resins is known under the name “MDTQ”, the resin being described as a function of the various siloxane monomer units it comprises, each of the letters M, D, T and Q characterizing a type of unit.

As examples of commercially available polymethylsilsesquioxane resins, mention may be made of those sold:
by the company Wacker under the reference Resin MK, such as Belsil PMS MK:
by the company Shin-Etsu under the reference KR-220L.

Siloxysilicate resins that may be mentioned include trimethyl siloxysilicate (TMS) resins such as those sold under the reference SR 1000 by the company General Electric or under the reference TMS 803 by the company Wacker. Mention may also be made of trimethyl siloxysilicate resins sold in a solvent such as cyclomethicone, sold under the name KF-7312J by the company Shin-Etsu or DC 749 and DC 593 by the company Dow Corning.

Mention may also be made of silicone resin copolymers such as those mentioned above with polydimethylsiloxanes, for instance the pressure-sensitive adhesive copolymers sold by the company Dow Corning under the reference Bio-PSA and described in document U.S. Pat. No. 5,162,410, or the silicone copolymers derived from the reaction of a silicone resin, such as those described above, and of a diorganosiloxane, as described in document WO 2004/073 626.

According to one embodiment of the invention, the film-forming polymer is a film-forming linear block ethylenic polymer, which preferably comprises at least one first block and at least one second block that have different glass transition temperatures (Tg), the said first and second blocks being linked together via an intermediate block comprising at least one constituent monomer of the first block and at least one constituent monomer of the second block.

Advantageously, the first and second blocks of the block polymer are mutually incompatible.

Such polymers are described, for example, in documents EP 1 411 069 or WO 04/028 488.

The lipophilic film-forming polymer may also be present in the composition in the form of particles dispersed in a non-aqueous phase. Examples of non-aqueous dispersions of film-forming polymers that may be mentioned include acrylic dispersions in isododecane, for instance Mexomer PAP® from the company Chimex, dispersions of particles of a grafted ethylenic polymer, which is preferably acrylic, in a liquid fatty phase, the ethylenic polymer advantageously being dispersed in the absence of additional surface stabilizer of particles, as described especially in document WO 04/055 081.

The composition according to the invention may comprise a plasticizer that promotes the formation of a film with the film-forming polymer. Such a plasticizer may be chosen from any compound known to those skilled in the art as being capable of satisfying the desired function.

Aqueous Phase

The composition may comprise an aqueous phase.

The aqueous phase comprises water. It may also comprise at least one water-soluble solvent. In the present invention, the term “water-soluble solvent” denotes a compound that is liquid at room temperature and water-miscible.

Preferably, the composition is anhydrous and more generally contains less than 10% by weight of water relative to the total weight of the composition, better still less than 5% by weight of water relative to the total weight of the composition, better still less than 2% and preferably less than 1% water.

Dyestuffs

The compositions in accordance with the invention preferably comprise at least one dyestuff such as pulverulent dyes, liposoluble dyes or water-soluble dyes.

The pulverulent dyestuffs may be chosen from pigments and nacres.

The pigments may be white or coloured, mineral and/or organic, and coated or uncoated. Among the mineral pigments, mention may be made of titanium dioxide, optionally surface-treated, zirconium, zinc or cerium oxide, and also iron or chromium oxide, manganese violet, ultramarine blue, chromium hydrate and ferric blue. Among the organic pigments that may be mentioned are carbon black, pigments of D & C type and lakes based on cochineal carmine or on barium, strontium, calcium or aluminium.

The nacres may be chosen from white nacreous pigments such as mica coated with titanium or with bismuth oxychloride, coloured nacreous pigments such as titanium mica with iron oxides, titanium mica especially with ferric blue or chromium oxide, titanium mica with an organic pigment of the abovementioned type, and also nacreous pigments based on bismuth oxychloride.

The liposoluble dyes are, for example, Sudan red, D&C Red 17, D&C Green 6, β-carotene, soybean oil, Sudan Brown, D&C Yellow 11, D&C Violet 2, D&C Orange 5, quinoline yellow and annatto.

These dyestuffs may be present in a content ranging from 0.01% to 30% by weight, relative to the total weight of the composition.

Fillers

The compositions in accordance with the invention may also comprise at least one filler.

The fillers may be selected from those that are well known to those skilled in the art and commonly used in cosmetic compositions. The fillers may be mineral or organic, and lamellar or spherical. Mention may be made of talc, mica, silica, kaolin, polyamide powders, for instance the Nylon® sold under the name Orgasol® by the company Atochem, poly-alanine powders and polyethylene powders, powders of tetra-fluoro-ethylene polymers, for instance Teflon®, lauroyllysine, starch, boron nitride, expanded polymeric hollow microspheres such as those of polyvinylidene chloride/-acrylonitrile, for instance the products sold under the name Expancel® by the company Nobel Industrie, acrylic powders such as those sold under the name Polytrap® by the company Dow Corning, polymethyl methacrylate particles and silicone resin microbeads (for example Tospearls® from Toshiba), precipitated calcium carbonate, magnesium carbonate and magnesium hydrocarbonate, hydroxyapatite, hollow silica microspheres (Silica Beads® from Maprecos), glass or ceramic microcapsules, metal soaps derived from organic carboxylic acids having from 8 to 22 carbon atoms and in particular from 12 to 18 carbon atoms, for example zinc, magnesium or lithium stearate, zinc laurate and magnesium myristate.

It is also possible to use a compound that is capable of swelling on heating, and especially heat-expandable particles such as non-expanded microspheres of vinylidene chloride/acrylonitrile/methyl methacrylate copolymer or of acrylonitrile homopolymer or copolymer, for instance those sold, respectively, under the references Expancel® 820 DU 40 and Expancel® 007WU by the company Akzo Nobel.

The fillers may represent from 0.1% to 25% by weight and in particular from 0.2% to 20% by weight relative to the total weight of the composition.

Fibres

The compositions in accordance with the invention may also comprise at least one fibre that can improve the lengthening effect.

The term “fibre” should be understood as meaning an object of length L and of diameter D such that L is very much greater than D, D being the diameter of the circle in which the cross section of the fibre is inscribed. In particular, the ratio L/D (or aspect ratio) is chosen in the range from 3.5 to 2500, in particular from 5 to 500 and more particularly from 5 to 150.

The fibres that may be used in the composition of the invention may be mineral or organic fibres, of synthetic or natural origin. They may be short or long, individual or organized, for example braided, and hollow or solid. They may have any shape and may especially have a circular or polygonal (square, hexagonal or octagonal) cross section depending on the specific application envisaged. In particular, their ends are blunted and/or polished to prevent injury.

In particular, the fibres have a length ranging from 1 μm to 10 mm, in particular from 0.1 mm to 5 mm and more particularly from 0.3 mm to 3.5 mm. Their cross section may be included in a circle with a diameter ranging from 2 nm to 500 μm, in particular ranging from 100 nm to 100 μm and more particularly from 1 μm to 50 μm. The weight or yarn count of fibres is often given in denier or decitex and represents the weight in grams per 9 km of yarn. The fibres according to the invention can in particular have a count chosen within the range from 0.15 to 30 denier and especially from 0.18 to 18 denier.

The fibres that may be used in the composition of the invention may be chosen from rigid or non-rigid fibres, and may be mineral or organic fibres, of synthetic or natural origin.

Moreover, the fibres may or may not be surface-treated, may be coated or uncoated, and may be coloured or uncoloured.

As fibres that may be used in the composition according to the invention, mention may be made of non-rigid fibres such as polyamide (Nylon®) fibres or rigid fibres such as polyimideamide fibres, for instance those sold under the names Kermel and Kermel Tech by the company Rhodia or poly(p-phenyleneterephthalamide) (or aramid) fibres sold especially under the name Kevlar® by the company DuPont de Nemours.

The fibres may be present in the composition according to the invention in a content ranging from 0.01% to 10% by weight, in particular ranging from 0.1% to 5% by weight and more particularly ranging from 0.3% to 3% by weight relative to the total weight of the composition.

Cosmetic Active Agents

The compositions in accordance with the invention may also comprise at least one cosmetic active agent.

As cosmetic active agents that may be used in the compositions in accordance with the invention, mention may be made especially of antioxidants, preserving agents, fragrances, neutralizers, emollients, thickeners, coalescers, plasticizers, moisturizers, vitamins and sunscreens, and mixtures thereof.

Needless to say, a person skilled in the art will take care to select the optional additional ingredients and/or the amount thereof such that the advantageous properties of the composition according to the invention are not, or are not substantially, adversely affected by the envisaged addition.

Preferably, the composition according to the invention is a leave-in composition. Advantageously, the composition is a makeup composition and in particular a mascara.

The examples that follow are given as illustrations of the present invention, and shall not limit the scope thereof.

Assembly

The assembly for coating keratin fibres according to the invention may comprise an applicator suitable for applying the said composition and, where appropriate, a conditioning device suitable for receiving the said composition.

Applicator

The applicator comprises means for smoothing and/or separating keratin fibres, such as the eyelashes or the eyebrows, especially in the form of teeth, bristles or other reliefs.

The applicator is arranged to apply the composition to the eyelashes or the eyebrows, and may comprise, for example, a brush or a comb.

The applicator may also be used for finishing makeup, on a region of the eyelashes or eyebrows that is made up or charged with composition.

The brush may comprise a twisted core and bristles held between the turns of the core, or may be made in yet another way.

The comb is made, for example, as a single piece by moulding a plastic material.

The application member may be magnetic.

In certain embodiments, the application member is mounted at the end of a stem, which may be flexible, thus contributing towards improving the comfort on application.

Conditioning Device

The conditioning device comprises a container for housing the composition for coating keratin fibres. This composition may then be taken up in the container by immersing the applicator therein.

This applicator may be integrally fastened to a member for closing the container. This closing member may form a member for handling the applicator. This handling member may form a cap to be removably mounted on the said container by any suitable means, such as by screwing, click-fastening, force-fitting or the like. Such a container may thus reversibly house the said applicator.

This container may optionally be equipped with a drainer arranged to remove the excess product taken up by the applicator.

A process for applying the composition according to the invention to the eyelashes or the eyebrows may also include the following steps:
forming a deposit of the cosmetic composition on the eyelashes or the eyebrows,
leaving the deposit on the eyelashes or the eyebrows, so that it can dry.

It should be noted that, according to another embodiment, the applicator may form a product container. In such a case, a container may, for example, be provided in the handling member, and an internal channel may internally connect this handling member to the application members in relief.

Finally, it should be noted that the conditioning and application assembly may be in the form of a kit, the applicator and the conditioning device being able to be housed separately in the same conditioning article.

### EXAMPLES

In order to illustrate the subject of the invention, a mascara composition comprising a copolymer according to the invention is prepared, as follows.

Preparation of an Isobornyl Acrylate/Isobornyl Methacrylate Copolymer Dissolved in Isododecane:

300 g of isododecane are placed in a reactor and heated to 90° C. while regulating the reaction medium.

At 90° C., a homogeneous mixture composed of 75 g of isobornyl acrylate, 225 g of isobornyl methacrylate and 3 g of Trigonox 141 is poured in over 1 hour.

The mixture is left stirring for 4 hours at 90° C.

The resulting mixture is diluted with 100 ml of isododecane and heated to 140° C. while regulating on an oil bath.

About 300 ml of isododecane are distilled off under vacuum (100 to 40 mbar) while simultaneously pouring in 200 ml of this same solvent. This operation is repeated twice.

A solution containing 40% polymer active material in isododecane is obtained.

The molar mass as PS equivalent in THF of the polymer solids obtained is 74 600 with Mn=38 600 and Mw=92 500.

| Ingredients | Content % |
|---|---|
| Isobornyl acrylate/isobornyl methacrylate copolymer as a 40% | 60 |
| solution in isododecane | |
| Isododecane (Ineos) | 34 |
| Black iron oxide (Sunpuro Black Iron Oxide C33-7001 - Sun) | 3 |
| Distearyldimethylammonium-modified hectorite (Bentone 38 | 3 |
| VCG from Elementis) | |

The ingredients of the composition are mixed together. The mixture is stirred with a blender of Rayneri type for 10 minutes.

The composition thus prepared is taken up using a mascara brush. The said composition is applied to a specimen of false eyelashes in two application passages. Curling of the eyelashes is then observed after drying of the deposit.

Besides this preferred implementation example of a copolymer in accordance with the invention comprising a copolymer obtained from 25% by weight of isobornyl acrylate monomers and 75% by weight of isobornyl methacrylate monomers, another advantageous implementation example may use a copolymer prepared from 50% by weight of isobornyl acrylate monomers and 50% by weight of isobornyl methacrylate monomers.

Throughout the application, the wording “comprising one” or “including one” means “comprising at least one” or “including at least one”, unless otherwise specified.

1. A cosmetic composition for coating keratin fibres, comprising:
a polymer, derived from a (co-) or (homo-)polymerization of (poly)cyclic monomers, and
a volatile organic solvent in a content of greater than 20% by weight relative to a total weight of the cosmetic composition,
wherein:
the polymer has a glass transition temperature Tg of greater than or equal to 80° C. and a weight-average molecular mass Mw of greater than 80 000 g/mol; and
the (poly)cyclic monomers comprise (meth)acrylate monomers of formula H₂C═C(R)—C(O)—O—X, wherein
R represents H or CH₃, and
X represents at least one substituted or unsubstituted cycloalkyl group.

2. The cosmetic composition according to claim 1, wherein the polymer is sterically hindered.

3. The cosmetic composition according to claim 1, wherein the at least one cycloalkyl group X of the (poly)cyclic monomers is substituted with at least one hydrocarbon-based group.

4. The cosmetic composition according to claim 3, wherein the at least one hydrocarbon-based group comprises from 1 to 6 carbon atoms.

5. The cosmetic composition according to claim 1, wherein the at least one cycloalkyl group X of the (poly)cyclic monomers is selected from the group consisting of an isobornyl, a dicyclopentyl, a dicyclopentenyl, an adamantyl, and a cyclohexyl.

6. The cosmetic composition according to claim 1, wherein the at least one cycloalkyl group X of the (poly)cyclic monomers is bridged.

7. The cosmetic composition according to claim 1, wherein the at least one cycloalkyl group X of the (poly)cyclic monomers comprises an unsaturation.

8. The cosmetic composition according to claim 1, wherein the polymer has a glass transition temperature Tg of greater than or equal to 100° C.

9. The cosmetic composition according to claim 1, wherein the polymer has a weight-average molecular mass of from 90 000 g/mol to 500 000 g/mol.

10. The cosmetic composition according to claim 1, wherein the polymer is free of hydrophilic monomer.

11. The cosmetic composition according to claim 1, wherein the polymer is soluble in the volatile organic solvent.

12. The cosmetic composition according to claim 1, wherein the polymer is a film-forming polymer.

13. The cosmetic composition according to claim 1, wherein the polymer is amorphous.

14. The cosmetic composition according to claim 1, wherein the polymer is a copolymer of isobornyl acrylate and isobornyl methacrylate.

15. The cosmetic composition according to claim 1, wherein the polymer is in a content of greater than or equal to 20% by weight relative to the total weight of the cosmetic composition.

16. The cosmetic composition according to claim 1, wherein the volatile organic solvent is hydrocarbon-based and comprises from 7 to 16 carbon atoms.

17. The cosmetic composition according to claim 1, wherein the cosmetic composition has a water content of less than or equal to 10% by weight relative to the total weight of the composition.

18. The cosmetic composition according to claim 1, wherein the cosmetic composition is a mascara.

19. A process for curling keratin fibres, comprising: applying the cosmetic composition according to claim 1.
